SEQUENCE LISTING (4 sequences)

SEQ ID NO: 1 - protein, 467 aa, Mus musculus
MGWTLVFLFLLSVTAGVHSQVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSV

SEQ ID NO: 3 - protein, 239 aa, Mus musculus
MVSSAQFLGLLLLCFQGTRCDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 112 aa, Mus musculus
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIK

(19) United States
(12) Patent Application Publication
(10) Pub. No.: US 2020/0150131 A1
(43) Pub. Date: May 14, 2020

(54) DIAGNOSTIC ADVANCED GLYCATION END-PRODUCT ANTIBODIES

(71) Applicant: Siwa Corporation, Chicago, IL (US)

(72) Inventor: Lewis S. Gruber, Chicago, IL (US)

(21) Appl. No.: 16/610,473

(22) PCT Filed: May 3, 2018
(86) PCT No.: PCT/US2018/030931

§ 371 Date: Nov. 1, 2019

Related U.S. Application Data
(60) Provisional application No. 62/501,424, filed on May 4, 2017.
(60) Provisional application No. 62/610,003, filed on Dec. 22, 2017.

(51) Int. Cl. G01N 33/68; G01N 33/574; A61K 49/00

(52) U.S. Cl. CPC G01N 33/6893; G01N 2800/7042; A61K 49/0002; G01N 33/574

(57) ABSTRACT

A method of diagnosing a disease, disorder or pathological condition associated with cellular senescence in a patient comprises obtaining a sample from the patient; measuring the number of cells that exhibit cell-surface AGEs in the sample; and diagnosing the patient with a disease, disorder or pathological condition associated with cellular senescence when the number of cells that exhibit cell-surface AGEs in the sample is greater than the number of cells that exhibit cell-surface AGEs in a control.

### BACKGROUND

Senescent cells are cells that are partially-functional or non-functional and are in a state of proliferative arrest. Senescence is a distinct state of a cell, and is associated with biomarkers, such as activation of the biomarker p16^{lnk4a}, and expression of β-galactosidase. Senescence begins with damage or stress (such as overstimulation by growth factors) of cells.

Advanced glycation end-products (AGEs; also referred to as AGE-modified proteins, or glycation end-products) arise from a non-enzymatic reaction of sugars with protein side-chains (Ando, K. et al., Membrane Proteins of Human Erythrocytes Are Modified by Advanced Glycation End Products during Aging in the Circulation, Biochem Biophys Res Commun., Vol. 258, 123, 125 (1999)). This process begins with a reversible reaction between the reducing sugar and the amino group to form a Schiff base, which proceeds to form a covalently-bonded Amadori rearrangement product. Once formed, the Amadori product undergoes further rearrangement: to produce AGEs. Hyperglycemia and oxidative stress promote this post-translational modification of membrane proteins (Lindsey J B, et al., “Receptor For Advanced Glycation End-Products (RAGE) and soluble RAGE (sRAGE): Cardiovascular Implications,” Diabetes Vascular Disease Research, Vol. 6(1), 7-14, (2009)). AGEs may also be formed from other processes. For example, the advanced glycation end product, N^{ε}-(carboxymethyl)lysine, is a product of both lipid peroxidation and glycoxidation reactions. AGEs have been associated with several pathological conditions including inflammation, retinopathy, nephropathy, atherosclerosis, stroke, endothelial cell dysfunction, and neurodegenerative disorders (Bierhaus A, “AGEs and their interaction with AGE-receptors in vascular disease and diabetes mellitus. I. The AGE concept,” Cardiovasc Res, Vol. 37(3), 586-600 (1998)).

AGE-modified proteins are also a marker of senescent cells. This association between glycation end-product and senescence is well known in the art. See, for example, Gruber, L. (WO 2009/143411, 26 Nov. 2009), Ando, K. et al. (Membrane Proteins of Human Erythrocytes Are Modified by Advanced Glycation End Products during Aging in the Circulation, Biochem Biophys Res Commun., Vol. 258, 123, 125 (1999)), Ahmed, E. K. et al. (“Protein Modification and Replicative Senescence of WI-38 Human Embryonic Fibroblasts” Aging Cells, vol. 9, 252, 260 (2010)), Vlassara, H. et al. (Advanced Glycosylation Endproducts on Erythrocyte Cell Surface Induce Receptor-Mediated Phagocytosis by Macrophages, J. Exp. Med., Vol. 166, 539, 545 (1987)) and Vlassara et al. (“High-affinity-receptor-mediated Uptake and Degradation of Glucose-modified Proteins: A Potential Mechanism for the Removal of Senescent Macromolecules” Proc. Natl. Acad. Sci. USAI, Vol. 82, 5588, 5591 (1985)). Furthermore, Ahmed, E. K. et al. indicates that glycation end-products are “one of the major causes of spontaneous damage to cellular and extracellular proteins” (Ahmed, E. K. et al., see above, page 353). Accordingly, the accumulation of glycation end-prod ucts is associated with senescence and lack of function.

The damage or stress that causes cellular senescence also negatively impacts mitochondrial DNA in the cells to cause them to produce free radicals which react with sugars in the cell to form methyl glyoxal (MG). MG in turn reacts with proteins or lipids to generate advanced glycation end products. In the case of the protein component lysine, MG reacts to form carboxyethyllysine, which is an AGE. (Al-Abed, Y. et al., “N^{ε}-Carboxymethyllysine formation by direct addition of glyoxal to lysine during the Maillard reaction”, Bioorganic & Medicinal Chemistry Letters Vol. 5, No. 18, pp. 2161-2162 (1995)).

Damage or stress to mitochondrial DNA also sets off a DNA damage response which induces the cell to produce cell cycle blocking proteins. These blocking proteins prevent the cell from dividing. Continued damage or stress causes mTOR production, which in turn activates protein synthesis and inactivates protein breakdown. Further stimulation of the cells leads to programmed cell death (apoptosis).

p16 is a protein involved in regulation of the cell cycle, by inhibiting the S phase (synthesis phase). It can be activated during aging or in response to various stresses, such as DNA damage, oxidative stress or exposure to drugs. p16 is typically considered a tumor suppressor protein, causing a cell to become senescent in response to DNA damage and irreversibly preventing the cell from entering a hyperproliferative state. However, there has been some ambiguity in this regard, as some tumors show overexpression of p16, while other show downregulated expression. Evidence suggests that overexpression of p16 is some tumors results from a defective retinoblastoma protein (“Rb”). p16 acts on Rb to inhibit the S phase, and Rb downregulates p16, creating negative feedback. Defective Rb fails to both inhibit the S phase and downregulate p16, thus resulting in overexpression of p16 in hyperproliferating cells (Romagosa, C. et al., p16^{lnk4a} overexpression in cancer: a tumor suppressor gene associated with senescence and high-grade tumors, Oncogene, Vol. 30, 2087-2097 (2011)).

Senescent cells are associated with secretion of many factors involved in intercellular signaling, including pro-inflammatory factors; secretion of these factors has been termed the senescence-associated secretory phenotype, or SASP (Freund, A. “Inflammatory networks during cellular senescence: causes and consequences” Trends Mol Med. 2010 May; 16(5):238-46). Autoimmune diseases, such as Crohn's disease and rheumatoid arthritis, are associated with chronic inflammation (Ferraccioli, G. et al. “Interleukin-1β and Interleukin-6 in Arthritis Animal Models: Roles in the Early Phase of Transition from Acute to Chronic Inflammation and Relevance for Human Rheumatoid Arthritis” Mol Med. 2010 November-December; 16(11-12): 552-557). Chronic inflammation may be characterized by the presence of pro-inflammatory factors at levels higher than baseline near the site of pathology, but lower than those found in acute inflammation. Examples of these factors include TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin. Senescent cells also upregulate genes with roles in inflammation including IL-1β, IL-8, ICAM1, TNFAP3, ESM1 and CCL2 (Burton, D. G. A. et al., “Microarray analysis of senescent vascular smooth muscle cells: a link to atherosclerosis and vascular calcification”, Experimental Gerontology, Vol. 44, No. 10, pp. 659-665 (October 2009)). Because senescent cells produce pro-inflammatory factors, removal of these cells alone produces a profound reduction in inflammation as well as the amount and concentration of pro-inflammatory factors.

Senescent cells secrete reactive oxygen species (“ROS”) as part of the SASP. ROS is believed to play an important role in maintaining senescence of cells. The secretion of ROS creates a bystander effect, where senescent cells induce senescence in neighboring cells: ROS create the very cellular damage known to activate p16 expression, leading to senescence (Nelson, G., A senescent cell bystander effect: senescence-induced senescence, Aging Cell, Vo. 11, 345-349 (2012)). The p16/Rb pathway leads to the induction of ROS, which in turn activates the protein kinase C delta creating a positive feedback loop that further enhance ROS, helping maintain the irreversible cell cycle arrest; it has even been suggested that exposing cancer cells to ROS might be effective to treat cancer by inducing cell phase arrest in hyperproliferating cells (Rayess, H. et al., Cellular senescence and tumor suppressor gene p16, Int J Cancer, Vol. 130, 1715-1725 (2012)).

The relative level of senescent cells has been specifically correlated with disease. Senescent cells have long been associated with cancer and metastatic cancer, and a cell culture with 10% senescent fibroblasts demonstrated growth stimulation (Krtolica, A. et al., “Senescent fibroblasts promote epithelial cell growth and tumorigenesis: a link between cancer and aging”, Proceedings of the National Academy of Sciences, Vol. 98, No. 21, pp. 12072-12077 (2001)). Aerobic fitness, a measure of biological aging, has been associated with 37% less senescent CD4+ and CD8+ T-cells (Spielmann, G. et al., “Aerobic fitness is associated with lower proportions of senescent blood T-cells in man”, Brain, Behavior and Immunity, Vol. 25, No. 8, pp. 1521-1529 (2011)). Likewise, senescent CD4+ T-cells increased 192% in triathletes two weeks after a 6-month training period for an Ironman triathlon (Cosgrove, C. et al., “The impact of 6-month training preparation for an Ironman triathlon on the proportions of naïve, memory and senescent T cells in resting blood”, European Journal of Applied Physiology, Vol. 112, No. 8, pp. 2989-2998 (2012)).

Increased levels of advanced glycation end-products, which are expressed on the surface of senescent cells, have been recognized as markers of various diseases, disorders and pathological conditions. Increased levels of carboxymethyllysine (CML) has been found in the serum and muscular tissue of fibromyalgia patients (Rüster, M. et al., “Detection of elevated N^{ε}-carboxymethyllysine levels in muscular tissue and in serum of patients with fibromyalgia”, Scandinavian Journal of Rheumatology, Vol. 34, No. 6, pp. 460-463 (2005)). Accelerated skin aging, such as thinner or wrinkled skin, is readily noticeable in diabetics (Schmid, D. et al., “Collage glycation and skin aging”, Cosmetics and Toiletries Manufacture Worldwide). Similarly, skin autofluorescence in diabetics is correlated with tissue levels of pentosidine and CML, and is strongly related to coronary heart disease and predicted mortality (Meerwaldt, R. et al., “Skin autofluorescence is a strong predictor of cardiac mortality in diabetes”, Diabetes Care, Vol. 30, No. 1, pp. 107-112 (2007)). Thus, elevated AGE levels are an accepted marker of diseases, disorders and pathological conditions associated with cellular senescence.

The absolute value of AGEs in a sample has been specifically correlated with disease. Levels of plasma CML were 30 μg/mL higher in patients with prostate cancer as compared to controls (Yang, S. et al., “Impact of oxidative stress biomarkers and carboxymethyllysine (an advanced glycation end product) on prostate cancer: a prospective study”, Clinical Genitourinary Cancer, Vol. 13, No. 5, pp. e347-e351 (2015)).

Similarly, the correlation between senescent cells and aging or age-related disorders, as described in WO 2009/143411, has resulted in age-related markers becoming diagnostic targets. Telomeres have long been associated with biological aging, and short telomere length has been used as an indicator of early-onset of age-related diseases such as diabetes, cardiovascular disease and cancer (“Telomere Testing White Paper”, Titanovo). Telomere length may be measured using polymerase chain reaction (PCR) analysis on DNA samples. A significant limitation of using telomere length to detect aging and age-related disorders is that not all senescence involves telomeres.

### SUMMARY

In a first aspect, the invention is a method of diagnosing a disease, disorder or pathological condition associated with cellular senescence in a patient comprising obtaining a sample from the patient; measuring the number of cells that exhibit cell-surface AGEs in the sample; and diagnosing the patient with a disease, disorder or pathological condition associated with cellular senescence when the number of cells that exhibit cell-surface AGEs in the sample is greater than the number of cells that exhibit cell-surface AGEs in a control.

In a second aspect, the invention is a method of determining the biological age of a patient comprising obtaining a sample from a patient containing cells and non-cellular material; separating the cells from the non-cellular material; measuring the number of cells that exhibit cell-surface AGEs in the sample by contacting the cells with an anti-AGE antibody and detecting binding between cell-surface AGEs and the anti-AGE antibody; measuring the number of unbound AGEs in the sample by contacting the non-cellular material with an anti-AGE antibody and detecting binding between unbound AGEs and the anti-AGE antibody; and comparing the ratio of cell-surface AGEs to unbound AGEs in the sample.

In a third aspect, the invention is a method of diagnosing a disease, disorder or pathological condition associated with advanced biological aging due to cellular senescence in a patient comprising obtaining a sample from a patient containing cells and non-cellular material; separating the cells from the non-cellular material; measuring the number of cells that exhibit cell-surface AGEs in the sample by contacting the cells with an anti-AGE antibody and detecting binding between cell-surface AGEs and the anti-AGE antibody; measuring the number of unbound AGEs in the sample by contacting the non-cellular material with an anti-AGE antibody and detecting binding between unbound AGEs and the anti-AGE antibody; comparing the ratio of cell-surface AGEs to unbound AGEs in the sample to determine the biological age of the patient; and diagnosing the patient with a disease, disorder or pathological condition associated with advanced biological aging due to cellular senescence when the biological age of the patient exceeds the chronological age of the patient.

In a fourth aspect, the invention is a method of diagnosing a disease, disorder or pathological condition associated with advanced biological aging due to cellular senescence in a patient comprising obtaining a sample from the patient; measuring the number of cells that exhibit cell-surface AGEs in the sample; determining the biological age of the patient by comparing the number of cells that exhibit cell-surface AGEs in the sample to the number of cells that exhibit cell-surface AGEs in an age-matched control; and diagnosing the patient with a disease, disorder or pathological condition associated with advanced biological aging due to cellular senescence when the biological age of the patient is greater than the chronological age of the patient.

In a fifth aspect, the invention is a method of detecting AGE-modified cells in a subject in vivo comprising administering to the subject an anti-AGE antibody that has been labeled with a detectable label.

In a sixth aspect, the invention is a kit for detecting cells expressing cell surface advanced glycation end-products comprising an anti-AGE antibody, a control sample, and, optionally, a reagent that binds to the anti-AGE antibody.

In a seventh aspect, the invention is a method of treating a disease, disorder or pathological condition associated with cellular senescence in a patient comprising administering a therapeutically effective amount of a senescent cell removal agent to a patient in need thereof. The biological age of the patient exceeds the chronological age of the patient.

### Definitions

The term “peptide” means a molecule composed of 2-50 amino acids.

The term “protein” means a molecule composed of more than 50 amino acids.

The terms “advanced glycation end-product”, “AGE”, “AGE-modified protein or peptide” and “glycation end-product” refer to modified proteins or peptides that are formed as the result of the reaction of sugars with protein side chains that further rearrange and form irreversible cross-links. This process begins with a reversible reaction between a reducing sugar and an amino group to form a Schiff base, which proceeds to form a covalently-bonded Amadori rearrangement product. Once formed, the Amadori product undergoes further rearrangement to produce AGEs. AGE-modified proteins and antibodies to AGE-modified proteins are described in U.S. Pat. No. 5,702,704 to Bucala (“Bucala”) and U.S. Pat. No. 6,380,165 to Al-Abed et al. (“Al-Abed”). Glycated proteins or peptides that have not undergone the necessary rearrangement to form AGEs, such as N-deoxyfructosyllysine found on glycated albumin, are not AGEs. AGEs may be identified by the presence of AGE modifications (also referred to as AGE epitopes or AGE moieties) such as 2-(2-furoyl)-4(5)-(2-furanyl)-1H-imidazole (“FFI”); 5-hydroxymethyl-1-alkylpyrrole-2-carbaldehyde (“Pyrraline”); 1-alkyl-2-formyl-3,4-diglycosyl pyrrole (“AFGP”), a non-fluorescent model AGE; carboxymethyllysine; carboxyethyllysine; and pentosidine. ALI, another AGE, is described in Al-Abed.

An “anti-AGE antibody” or “AGE antibody” means an antibody, antibody fragment or other protein or peptide that binds to an AGE-modified protein or peptide, and preferably includes a constant region of an antibody. The AGE-modified protein or peptide may be a protein or peptide normally found bound on the surface of a cell, preferably a mammalian cell, more preferably a human, cat, dog, horse, camelid (for example, camel or alpaca), cattle, sheep, or goat cell. Alternatively, the AGE-modified protein or peptide may be a protein or peptide that is not bound to the surface of a cell (also referred to as free, unbound or circulating proteins or peptides). An “anti-AGE antibody” or “AGE antibody” does not include an antibody or other protein which binds with the same specificity and selectivity to both the AGE-modified protein or peptide, and the same non-AGE-modified protein or peptide (that is, the presence of the AGE modification does not increase binding). An “anti-AGE antibody” or “AGE antibody” includes antibodies which are conjugated, for example to a toxin, drug, or other chemical or particle. Preferably, the antibodies are monoclonal antibodies, but polyclonal antibodies are also possible.

The term “senescent cell” means a cell which is in a state of proliferative arrest and expresses one or more biomarkers of senescence, such as activation of p16^{lnk4a} or expression of senescence-associated β-galactosidase. Also included are cells which express one or more biomarkers of senescence, do not proliferate in vivo, but may proliferate in vitro under certain conditions, such as some satellite cells found in the muscles of ALS patients.

The term “senolytic agent” means a small molecule with a molecular weight of less than 900 daltons that destroys senescent cells. The term “senolytic agent” does not include antibodies, antibody conjugates, proteins, peptides or biologic therapies.

The term “senescent cell removal agent” means a substance that destroys senescent cells. Senescent cell removal agents include therapeutic anti-AGE, antibodies such as those described in U.S. Pat. No. 9,161,810 and senolytic agents.

The term “variant” means a nucleotide, protein or amino acid sequence different from the specifically identified sequences, wherein one or more nucleotides, proteins or amino acid residues is deleted, substituted or added. Variants may be naturally-occurring allelic variants, or non-naturally-occurring variants. Variants of the identified sequences may retain some or all of the functional characteristics of the identified sequences.

The term “percent (%) sequence identity” is defined as the percentage of amino acid residues in a candidate sequence that are identical to the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using publicly available computer software such as BLAST, BLAST-2, ALIGN or Megalign (DNASTAR) software. Preferably, % sequence identity values are generated using the sequence comparison computer program ALIGN-2. The ALIGN-2 sequence comparison computer program is publicly available from Genentech, Inc. (South San Francisco, Calif.), or may be compiled from the source code, which has been filed with user documentation in the U.S. Copyright Office and is registered under U.S. Copyright Registration No. TXU510087. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained using the ALIGN-2 computer program.

### BRIEF DESCRIPTION OF THE DRAWING

FIG. 1 illustrates a kit for detecting cells expressing cell surface advanced glycation end-products.

FIG. 2 is a graph of the response versus time in an antibody binding experiment.

FIG. 3A is a photograph of cells of an Alzheimer's disease sample showing carboxymethyllysine stained red (dark gray) and phosphorylated tau stained green (light gray).

FIG. 3B is a photograph of cells of an Alzheimer's disease sample showing carboxymethyllysine stained red (dark gray) and amyloid precursor protein stained green (light gray).

FIG. 3C is a photograph of cells of a Parkinson's disease sample from the substantia nigra showing carboxymethyllysine stained red (dark gray) and alpha synuclein stained green (light gray).

FIG. 3D is a photograph of cells of a Parkinson's disease sample from the ventral tegmental area showing carboxymethyllysine stained red (dark gray) and alpha synuclein stained green (light gray).

### DETAILED DESCRIPTION

The recognition of a quantifiable association between cellular senescence and various diseases, disorders and pathological conditions has resulted in senescent cells becoming a diagnostic target. For example, CD57 is a known marker of senescent cells, including immune cells such as natural killer (NK) cells and T-cells (Kared, H. et al., “CD57 in human natural killer cells and T-lymphocytes”, Cancer Immunology, Immunotherapy, Vol. 65, No. 4, pp. 441-452 (2016)). CD57 isolation kits are commercially available, such as the CD8+CD57+ T Cell Isolation Kit from Miltenyi Biotec (Bergisch Gladbach, Germany), but these quantitative measurement tools are intended for research use only. The data sheet for the Miltenyi Biotec T Cell isolation kit explicitly states that the kit is not for diagnostic or therapeutic use.

The detection and quantification of advanced glycation end-products has been carried out with analytical techniques that are capable of detecting proteins, such as mass spectrometry and high-performance liquid chromatography. However, these techniques are cumbersome and often rely on complex laboratory equipment. Wet lab techniques for measurement of advanced glycation end-products using anti-AGE antibodies, such as immunoassays, are significantly easier to use. Anti-AGE assays are commercially available, such as the Carboxymethyl Lysine (CML) ELISA Cat. No. KT-32428 from Kamiya Biomedical Company (Seattle, Wash., USA). Much like the senescent cell detection tools, these quantitative measurement tools are intended for research use only. The data sheet for the Kamiya Biomedical Company CML ELISA (Cat. No. KT-32428) explicitly states that the product is not for use in diagnostic procedures. Accordingly, the use of anti-AGE antibodies for diagnostic purposes is neither routine nor conventional.

The present invention uses antibodies that bind to advanced glycation end-product-modified proteins and peptides to diagnose and monitor senescence-associated diseases, disorders or pathological conditions. AGE-modified proteins and peptides, especially AGE-modified proteins and peptides on the surface of partially-functional and non-functional cells, are a unique target for antibody-based diagnostic methods including the enzyme-linked immunosorbent assay (ELISA), cell sorting and cell counting. For example, detection of cell-bound carboxymethyllysine (CML), a well-known advanced glycation end-product, may be used to determine the total number, concentration or ratio of senescent cells in a sample. Patients may be identified as in need of treatment based on the number of cells that exhibit cell-surface AGEs in a sample as compared to the number of cells that exhibit cell-surface AGEs in a control, or when the number of cells that exhibit cell-surface AGEs in the sample exceeds a clinical threshold. Alternatively, or in addition, comparing the ratio of cell-bound AGEs to free (unbound) AGEs may be used to normalize the number of senescent cells in the sample and monitor disease progression or biological aging. A greater ratio of cell-bound AGEs indicates a greater amount of cellular senescence due to internal sources and cell dysfunction. Anti-AGE antibody-based diagnostic methods offer the advantages of being minimally invasive and simple to carry out, which allows such tests to be carried out in a doctor's office or clinic.

An anti-AGE antibody may be used to detect the presence of senescent cells in a sample since senescent cells express cell-surface advanced glycation end-products. In one embodiment, a sample is provided. The sample may be obtained from a human patient. Next, the presence of cell-surface AGEs in the sample is determined or measured by contacting the sample with an anti-AGE antibody and detecting binding between cell-surface AGEs and the anti-AGE antibody. Optionally, a control sample may be obtained from the patient, or from a healthy subject, as a baseline for comparison. A baseline for comparison may also be obtained as an average number of cells exhibiting an AGE-modification from a group of healthy controls. Preferably, the control samples are obtained from healthy subjects that are the same chronological age as the patient from whom the sample is obtained (also known as “age-matched” or “age-indexed” controls).

The number of cells exhibiting cell-surface AGEs may be determined using qualitative or quantitative measurements. The measurement is intended to provide information that is useful for comparison to a healthy control. Examples of quantitative measurements include measuring the total number, average number, concentration, ratio or percentage of cells exhibiting cell-surface AGEs in a sample. Examples of qualitative measurements include analyzing tissue samples with immunohistochemical or immunocytochemical techniques. For example, the location of glycation within a sample may be indicative of a disease, disorder or pathological condition associated with cellular senescence.

The measurement of senescent cells in the sample may be used to diagnose the patient with a disease, disorder or pathological condition associated with cellular senescence, or to identify the patient as in need of treatment. An elevated level of cellular dysfunction may be indicated when the total number, average number, concentration, ratio or percentage of cells that exhibit cell-surface AGEs in the sample exceeds the total number, average number, concentration, ratio or percentage of cells that exhibit cell-surface AGEs in a control. For example, a sample that contains greater than 5% senescent cells is indicative of an elevated level of cellular dysfunction. A patient may be diagnosed with a disease, disorder or pathological condition associated with cellular senescence or identified as in need of treatment prior to demonstrating symptoms or receiving a clinical diagnosis from a health care professional. Preferably, the patient already exhibits at least one symptom of the disease, disorder or pathological condition associated with cellular senescence prior to testing to aid in identification of the disease, disorder or pathological condition associated with cellular senescence. The measurement of senescent cells in the sample may also be used in differential diagnosis to distinguish diseases, disorders or pathological conditions with overlapping or similar symptoms.

An elevated level of cellular dysfunction may also be indicated when the number of cells that exhibit cell-surface AGEs in the sample exceeds a clinical threshold. For example, a patient may be diagnosed with a disease, disorder or pathological condition associated with cellular senescence or identified as in need of treatment if the sample contains 5%-50% senescent cells, including at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40% and 45% senescent cells. The clinical threshold may be based on the highest number or the average number of AGE-modified cells found in a collection of samples obtained from healthy patients.

The measurement of senescent cells in the sample may also be used 1:0 determine the biological age of the patient. Since senescent cells accumulate with age, a greater number of senescent cells in a sample as compared to an age-matched control is indicative of an advanced biological age. A patient may be identified as in need of treatment when the biological age of the patient is greater than the chronological age of the patient. For example, a patient may be diagnosed with advanced biological age when her biological age is 10%-50% greater than her chronological age, including at least 15%, 20%, 25%, 30%, 35%, 40% and 45% greater than her chronological age. Similarly, a patient may be diagnosed with advanced biological age when her biological age is 5-50 years greater than her chronological age, including at least 10 years, 15 years, 20 years, 25 years, 30 years, 35 years, 40 years and 45 years greater than her chronological age.

An anti-AGE antibody may also be used to detect free (unbound) AGEs and AGE-modified proteins or peptides in the sample. The free AGEs may serve as a measure of advanced glycation end-products that are not associated with cellular senescence. The number of cell-surface AGEs in the sample may be compared to the number of free AGEs to normalize the number of senescent cells in the sample. For example, the ratio of cell-surface AGEs to free AGEs may be used to determine the percentage AGEs that have accumulated on the cell surface, with a high ratio indicating an increase in cellular senescence. The percentage of senescent cells in a sample may also be used as a measure of the biological age of the patient.

The sample may be any substance obtained from the patient that contains cells which may be senescent. Examples of suitable samples include saliva, a buccal swab, a blood sample, a urine sample, a skin sample and a biopsy. The sample may optionally be physically processed, such as by centrifugation, or chemically processed, such as by trypsinization. Sample processing may be used to isolate specific portions of the sample, such as separating a blood sample into serum and plasma.

The cells within the sample being tested for the presence of cell-surface advanced glycation end-products may be any cells that are capable of undergoing cellular senescence. Examples of suitable cells to be tested include T-cells, erythrocytes, fibroblasts and epithelial cells. T-cells are a preferred cell for testing.

The presence of AGE-modified peptides or proteins in the sample may be determined by any antibody-based identification technique. Examples of suitable antibody identification techniques include immunoassays, such as enzyme-linked immunosorbent assays (ELISAs), radioimmunoassays (RIAs) and real-time immunoquantitative PCR (iqPCR), cell sorting, such as fluorescent activated cell sorting (FACS), flow cytometry and magnetic cell sorting, cell counting, Western blots, immunohistochemistry (IHC), immunocytochemistry (ICC), immunoprecipitation and enzyme linked immunospot (ELISPOT). Preferably, the antibody identification technique is an immunoassay.

A preferred technique for detecting senescent cells in tissue samples is immunohistochemical (IHC) staining. Histological analysis of tissue samples is a well-established technique for identification of specific proteins in a tissue sample. For example, AGEs have been detected in tissue samples of atherosclerotic lesions and pancreatic cancer (Wendel, U. et al., “A novel monoclonal antibody targeting carboxymethyllysine, an advanced glycation end product in atherosclerosis and pancreatic cancer”, PLoS One, Vol. 13, No. 2, e0191872 (2018)). Immunohistochemical staining allows for detecting specific sites of glycation.

The diagnostic techniques may be carried out on-site where the sample was obtained. Alternatively, the sample may be sent to an off-site testing facility, such as a laboratory.

The anti-AGE antibody may be any antibody that binds to an AGE-modified protein or peptide, including AGE-modified proteins or peptides that are expressed on the surface of senescent cells. Anti-AGE antibodies are known in the art and are commercially available. Examples include those described in U.S. Pat. No. 5,702,704 (Bucala) and U.S. Pat. No. 6,380,165 (Al-Abed et al.). The antibody may bind to one or more AGE-modified proteins or peptides having an AGE modification such as FFI, pyrraline, AFGP, ALI, carboxymethyllysine (CML), carboxyethyllysine (CEL) and pentosidine, and mixtures of such antibodies. The antibody may be monoclonal or polyclonal. Preferably, the antibody is a monoclonal antibody.

Preferred anti-AGE antibodies include those which bind to proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents). A preferred commercially-available anti-AGE antibody is the mouse anti-glycation end-product antibody raised against carboxymethyl lysine conjugated with keyhole limpet hemocyanin (Clone 318003) available from R&D Systems, Inc. (Minneapolis, Minn.; catalog no. MAB3247).

An anti-AGE antibody may have or may include a heavy chain having the protein sequence of SEQ ID NO: 1 and a light chain having the protein sequence of SEQ ID NO: 3. The variable domains of the heavy chain and the light chain are shown in SEQ ID NO: 2 and SEQ ID NO: 4, respectively. The DNA and protein sequences of additional anti-AGE antibodies may be found in WO 2017/143073, the publication of International Patent Application No. PCT/US2017/18185, which is herein incorporated by reference.

The anti-AGE antibody may optionally be a bi-specific antibody, which is an antibody directed to two different epitopes. Such antibodies include a variable region (or complementary determining region) from one anti-AGE antibody, and a variable region (or complementary determining region) from a different antibody.

Antibody fragments may be used in place of whole antibodies. For example, immunoglobulin G may be broken down into smaller fragments by digestion with enzymes. Papain digestion cleaves the N-terminal side of inter-heavy chain disulfide bridges to produce Fab fragments. Fab fragments include the light chain and one of the two N-terminal domains of the heavy chain (also known as the Fd fragment). Pepsin digestion cleaves the C-terminal side of the inter-heavy chain disulfide bridges to produce F(ab′)₂ fragments. F(ab′)2 fragments include both light chains and the two N-terminal domains linked by disulfide bridges. Pepsin digestion may also form the Fv (fragment variable) and Fc (fragment crystallizable) fragments. The Fv fragment contains the two N-terminal variable domains. The Fc fragment contains the domains which interact with immunoglobulin receptors on cells and with the initial elements of the complement cascade. Pepsin may also cleave immunoglobulin G before the third constant domain of the heavy chain (C_{H}3) to produce a large fragment F(abc) and a small fragment pFc′. Antibody fragments may alternatively be produced recombinantly.

Antibodies may be produced using well-known methods. For example, polyclonal antibodies (pAbs) can be raised in a mammalian host by one or more injections of an immunogen, and if desired, an adjuvant. Typically, the immunogen (and adjuvant) is injected in a mammal by a subcutaneous or intraperitoneal injection. The immunogen may be an AGE-modified protein or peptide of a cell, such as AGE-antithrombin III, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-RNAse, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Examples of adjuvants include Freund's complete, monophosphoryl Lipid A synthetic-trehalose dicorynomycolate, aluminum hydroxide (alum), heat shock proteins HSP 70 or HSP96, squalene emulsion containing monophosphoryl lipid A, α2-macroglobulin and surface active substances, including oil emulsions, pleuronic polyols, polyanions and dinitrophenol. To improve the immune response, an immunogen may be conjugated to a polypeptide that is immunogenic in the host, such as keyhole limpet hemocyanin (KLH), serum albumin, bovine thyroglobulin, cholera toxin, labile enterotoxin, silica particles or soybean trypsin inhibitor. A preferred immunogen conjugate is AGE-KLH. Alternatively, pAbs may be made in chickens, producing IgY molecules.

Monoclonal antibodies (mAbs) may be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Other techniques may be used, such as the EBV-hybridoma technique. If desired, the mAbs may be purified from the culture medium or ascites fluid by conventional procedures, such as protein A-sepharose, hydroxyapatite chromatography, gel electrophoresis, dialysis, ammonium sulfate precipitation or affinity chromatography.

The anti-AGE antibodies may be used to diagnose the onset or measure the progression of any disease, disorder or pathological condition that is characterized by cellular senescence. Examples of diseases, disorders and pathological conditions that have been associated with cellular senescence include Alzheimer's disease, amyotrophic lateral sclerosis (ALS or Lou Gehrig's Disease), chronic obstructive pulmonary disease (COPD), Huntington's chorea, idiopathic pulmonary fibrosis, muscular dystrophy (including Becker's, Duchenne, Limb-Girdle and Yamamoto's muscular dystrophy), macular degeneration, cataracts, diabetic retinopathy, Parkinson's disease, progeria (including Werner Syndrome and Hutchinson Gilford progeria), vitiligo, cystic fibrosis, atopic dermatitis, eczema, arthritis (including osteoarthritis, rheumatoid arthritis and juvenile rheumatoid arthritis), atherosclerosis, cancer and metastatic cancer (including, for example, breast cancer, triple negative breast cancer, lung cancer, melanoma, colon cancer, renal cell carcinoma, prostate cancer, cancer of the cervix, bladder cancer, rectal cancer, esophageal cancer, liver cancer, mouth and throat cancer, multiple myeloma, ovarian cancer, stomach cancer, pancreatic cancer and retinal blastoma cancers), cancer therapy-related disability or cancer therapy side effects, hypertension, glaucoma, osteoporosis, sarcopenia, cachexia, stroke, myocardial infarction, atrial fibrillation, transplantation rejection, diabetes mellitus—Type I, diabetes mellitus—Type II, radiation exposure, HIV treatment side effects chemical weapons exposure, poisoning, inflammation, nephropathy, Lewy body dementia, prion disease (including bovine spongiform encephalopathy, Creutzfeldt-Jakob disease, scrapie, chronic wasting disease, kuru and fatal familial insomnia), lordokyphosis, auto-immune disorders, loss of adipose tissue, psoriasis, Crohn's disease, asthma, the physiological effects of aging (including “cosmetic” effects, such as wrinkling, age spots, hair loss, reduction in subcutaneous adipose tissue and thinning of the skin), idiopathic myopathy (including, for example, idiopathic inflammatory myopathy, idiopathic inflammatory myositis, polymyositis, dermatomyositis, sporadic inclusion body myositis and juvenile myositis), multiple sclerosis, neuromyelitis optica (NMO, Devic's disease or Devic's syndrome), epilepsy and adrenoleukodystrophy (ALD, X-linked adrenoleukodystrophy, X-ALD, cerebral ALD or cALD).

Any subject that could develop a disease, disorder or pathological condition associated with cellular senescence may be diagnosed by the methods herein described. The subject may be a mammal. Humans are a preferred subject for diagnosis. Other subjects that may be diagnosed include mice, rats, goats, sheep, cows, horses, camels and companion animals, such as dogs or cats.

A patient who has been diagnosed with a disease, disorder or pathological condition associated with cellular senescence or identified as in need of treatment may be administered a senescent cell removal agent to target and destroy senescent cells. Examples of senescent cell removal agents include therapeutic anti-AGE antibodies, an anti-AGE antibody conjugated to a toxin, a senolytic agent, such as dasatinib and/or quercetin, and combinations thereof. Senescent cells may also be destroyed by the application of therapeutic ultrasound. Senescent cell destruction techniques may be combined to achieve a desired therapeutic outcome. For example, a patient may be administered a combination of dasatinib and quercetin as well as high intensity focused ultrasound to selectively destroy senescent cells while sparing functional cells. A therapeutically effective amount of the senescent cell removal agent will vary depending on the specific senescent cell removal agent used. For example, an appropriate dosage level of an anti-AGE antibody will generally be about 0.01 to 500 mg/kg patient body weight, including about 0.01 to 250 mg/kg, about 0.05 to 100 mg/kg, and about 0.1 to 50 mg/kg. Similarly, an appropriate dosage level of the combination therapy dasatinib and quercetin will generally be about 5 mg/kg patent body weight dasatinib and about 50 mg/kg patent body weight quercetin. Treatment efficacy may be monitored by repeated measurements of the number of cells exhibiting cell-surface AGES.

Administration of a senescent cell removal agent has been demonstrated as effective in treating sarcopenia, atherosclerosis and metastatic cancer. Other diseases, disorders and pathological conditions associated with cellular senescence that are particularly suitable for treatment by administration of a senescent cell removal agent include inflammation, autoimmune diseases, osteoarthritis. Alzheimer's disease and Parkinson's disease.

In addition to the in vitro diagnostic methods described above, anti-AGE antibodies may also be used in in vivo diagnostic methods. In vivo diagnostic tests provide non-invasive methods of detecting AGE-modified proteins and peptides. In vivo diagnostic tests are particularly useful for detecting senescent cells that express cell-surface advanced glycation end-products, such as metastatic cancer cells. (See, for example, WO 2017/143073). Anti-AGE antibodies may be labeled with a detectable label or tracer and then administered to a subject. The labeled anti-AGE antibodies specifically bind to AGE-modified proteins or peptides, which allows the AGE-modified proteins or peptides to be detected with any suitable apparatus that is capable of detecting the label. Examples of in vivo diagnostic methods include positron emission tomography (PET) and immuno-PET, magnetic resonance imaging (MRI), single photon emission computed tomography (SPECT), optical imaging, ultrasound, radioimmunoscintigraphy and combinations thereof. Any label that is appropriate for a given diagnostic technique may be used, such as radiolabels, fluorescent labels, positron emitters, dyes that emit in near infrared (NIR), nanoparticles such as gold and gadolinium, quantum dots, superparamagnetic iron oxide (SPIO), carbon nanotubes or microbubbles that have been conjugated to the antibodies.

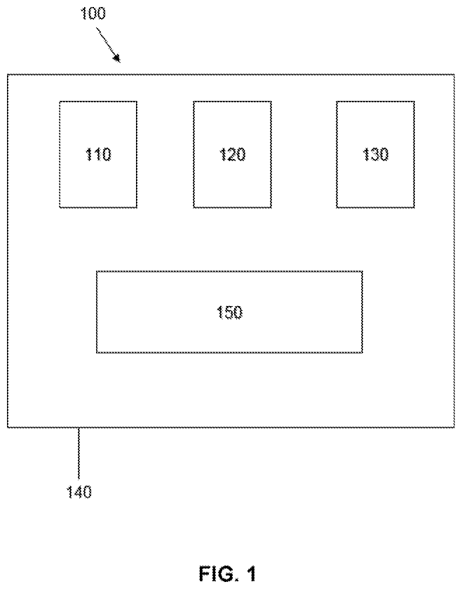

FIG. 1 illustrates a kit 100 for detecting cells expressing cell surface advanced glycation end-products. The kit may include an anti-AGE antibody 110, a control 120 and, optionally, a reagent 130 for detecting the anti-AGE antibody. The anti-AGE antibody, the control and the optional reagent may be supplied in any suitable container, such as bottles, ampules, envelopes, test tubes, vials, flasks or syringes. The anti-AGE antibody and/or the reagent may optionally be labelled, such as with a fluorescent label, radiolabel or a gold particle. The control may be normal serum from an animal in which a secondary antibody was made, a solution containing a known amount of an AGE-modified protein or peptide or fixed or preserved cells that exhibit and AGE modification. Examples of reagents for detecting the anti-AGE antibody include secondary antibodies, such as an anti-human polyclonal antibody made in donkey and labelled with rhodamine. The kit may optionally be housed in a container 140. The kit may optionally include printed instructions 150. Preferably, the contents of the kit are sterile and ready for use.

The kit may optionally include a container for housing the kit ingredients. The container may be formed of a rigid, durable material, such as plastic, or may be flexible, such as a bag or soft-sided box.

The kit may optionally include instructions for use. The instructions may be provided as printed instructions or in electronic format, such as on a universal serial bus (USB) drive, on a secure digital (SD) card, or hosted over the internet and accessible through a quick response (QR) code.

Kits may optionally contain additional diagnostic materials or equipment such as buffers, fixatives, blocking solutions, protease inhibitors, substrates for analysis such as microscope slides and/or cover slips, microtiter plates and cell extraction reagents such as detergents and detergent solutions.

### EXAMPLES

### Example 1: Collection of Buccal Epithelial Cells

A patient swishes a saline solution in her mouth for 30 seconds. She then spits the solution into a cup. 1.5 mL of the saline solution from the cup is then transferred to a centrifuge tube using a micropipette. The centrifuge tube is placed into a balanced centrifuge and centrifuged at 10,000-14,000 RPM for 2 minutes. The centrifuging may be repeated until a pellet is visible in the bottom of the centrifuge tube. The supernatant is then discarded by decanting and/or removing it with a micropipette. The pellet contains isolated buccal epithelial cells which may then be tested for the presence of cell-surface AGEs to determine if any of the epithelial cells are senescent.

### Example 2: Diagnosis and Treatment Based on Skin Cells

Epidermal cells are collected using a tape harvesting process. Adhesive tape (Adhesives Research, Glen Rock, Pa.) is fabricated into circular disks approximately 17 mm in diameter. The tape is applied to a patient's skin, then removed to harvest epidermal cells from the stratum corneum. Tape harvesting is repeated 3 additional times to obtain a total of 4 epidermal samples. The epidermal cells are then tested for the presence of cell-surface AGEs to determine if any of the epidermal cells are senescent.

Dermal cells are collected using a shave biopsy. A scalpel blade is used to remove sufficient skin to pass through the epidermis and access the dermis to obtain a sample. Fibroblasts within the sample are then tested for the presence of cell-surface AGEs to determine if any of the fibroblasts are senescent. The presence of at least 5% senescent cells in the shave biopsy indicates skin damage and the need for treatment to reduce the number of senescent skin cells. The patient is administered a senescent cell removal agent to target and remove senescent skin cells.

### Example 3: Direct Binding ELISA Using Anti-AGE Antibodies

The binding of murine and chimeric anti-AGE antibodies was investigated by a direct binding ELISA. An anti-carboxymethyl lysine (CML) antibody (R&D Systems, MAB3247) was used as a control. CML was conjugated to KLH (CML-KLH) and both CML and CML-KLH were coated overnight onto an ELISA plate. HRP-goat anti-mouse Fc was used to detect the control and murine anti-AGE antibodies. HRP-goat anti-human Fc was used to detect the chimeric anti-AGE antibody.

The antigens were diluted to 1 μg/mL in 1× phosphate buffer at pH 6.5. A 96-well microtiter ELISA plate was coated with 100 μL/well of the diluted antigen and let sit at 4° C. overnight. The plate was blocked with 1×PBS, 2.5% BSA and allowed to sit for 1-2 hours the next morning at room temperature. The antibody samples were prepared in serial dilutions with 1×PBS, 1% BSA with the starting concentration of 50 μg/mL. Secondary antibodies were diluted 1:5,000. 100 μL of the antibody dilutions was applied to each well. The plate was incubated at room temperature for 0.5-1 hour on a microplate shaker. The plate was washed 3 times with 1×PBS. 100 μL/well diluted HRP-conjugated goat anti-human Fc secondary antibody was applied to the wells. The plate was incubated for 1 hour on a microplate shaker. The plate was then washed 3 times with 1×PBS. 100 μL HRP substrate TMB was added to each well to develop the plate. After 3-5 minutes elapsed, the reaction was terminated by adding 100 μL of 1N HCl. A second direct binding ELISA was performed with only CML coating. The absorbance at OD450 was read using a microplate reader.

The OD450 absorbance raw data for the CML and CML-KLH ELISA is shown in the plate map below. 48 of the 96 wells in the well plate were used. Blank wells in the plate map indicate unused wells.

Plate Map of CML and CML-KLH ELISA:

| | Conc. (ug/mL) | | | | | | |
|---|---|---|---|---|---|---|---|
| | 1 | 2 | 3 | 4 | 5 | 6 | 7 |
| 50 | 0.462 | 0.092 | 0.42 | | 1.199 | 0.142 | 1.852 |
| 16.67 | 0.312 | 0.067 | 0.185 | | 0.31 | 0.13 | 0.383 |
| 5.56 | 0.165 | 0.063 | 0.123 | | 0.19 | 0.115 | 0.425 |
| 1.85 | 0.092 | 0.063 | 0.088 | | 0.146 | 0.099 | 0.414 |
| 0.62 | 0.083 | 0.072 | 0.066 | | 0.108 | 0.085 | 0.248 |
| 0.21 | 0.075 | 0.066 | 0.09 | | 0.096 | 0.096 | 0.12 |
| 0.07 | 0.086 | 0.086 | 0.082 | | 0.098 | 0.096 | 0.098 |
| 0 | 0.09 | 0.085 | 0.12 | | 0.111 | 0.083 | 0.582 |
| | R&D | Parental | Chimeric | | R&D | Parental | Chimeric |
| | Positive | Anti- | Anti- | | Positive | Anti- | Anti- |
| | Control | AGE | AGE | | Control | AGE | AGE |

| | |
|---|---|
| CML-KLH Coat | CM L Coat |

The OD450 absorbance raw data for the CML-only ELISA is shown in the plate map below. 24 of the 96 wells in the well plate were used. Blank wells in the plate map indicate unused wells.

Plate Map of CML-Only ELISA:

| | Conc. (ug/mL) | | | | | | |
|---|---|---|---|---|---|---|---|
| | 1 | 2 | 3 | 4 | 5 | 6 | 7 |
| 50 | 1.913 | 0.165 | 0.992 | | | | |
| 16.66667 | 1.113 | 0.226 | 0.541 | | | | |
| 5.555556 | 0.549 | 0.166 | 0.356 | | | | |
| 1.851852 | 0.199 | 0.078 | 0.248 | | | | |
| 0.617284 | 0.128 | 0.103 | 0.159 | | | | |
| 0.205761 | 0.116 | 0.056 | 0.097 | | | | |
| 0.068587 | 0.073 | 0.055 | 0.071 | | | | |
| 0 | 0.053 | 0.057 | 0.06 | | | | |
| | R&D | Parental | Chimeric | | | | |
| | Positive | Anti- | Anti- | | | | |
| | Control | AGE | AGE | | | | |

The control and chimeric anti-AGE antibodies showed binding to both CML and CML-KLH. The murine (parental) anti-AGE antibody showed very weak to no binding to either CML or CML-KLH. Data from repeated ELISA confirms binding of the control and chimeric anti-AGE antibody to CML. All buffer control showed negative signal.

This data confirms the ability of the anti-AGE antibodies to bind AGEs and AGE-immunogen conjugates. Evidence of binding to the well-known AGE carboxymethyllysine supports the suitability of anti-AGE antibodies in diagnostic applications.

### Example 4: Diagnosis and Treatment Based on Blood Sample

A blood sample is drawn from a patient. The blood sample is centrifuged to isolate the serum. CD57+ T-cells are isolated using the Miltenyi Biotec CD8+CD57+ T Cell Isolation Kit (Bergisch Gladbach, Germany). CD57+ T-cells are counted using a hemocytometer. The serum and the isolated CD57+ T-cells are then tested for binding to an anti-CML antibody. Serum CML less than or equal to 152 μg/mL combined with greater than or equal to 50% of isolated CD57+ T-cells binding to a labeled anti-CML antibody indicates that the patient is in need of treatment with a senescent cell removal agent. The patient is administered an anti-AGE antibody that targets and removes senescent cells.

### Example 5: Diagnosis and Treatment Based on Buccal Swab

A sample is obtained from a patient by a buccal swab. The buccal epithelial cells and the saliva from the swab are separated. The buccal cells are trypsinized and mixed with an anti-CML monoclonal antibody. The mixture is then passed through a hemocytometer to count the senescent buccal cells. Free CML is measured by an ELISA of the saliva. Saliva CML less than or equal to 3 μg/mL combined with greater than or equal to 50% of the buccal cells binding to the anti-CML antibody indicates the patient is in need of treatment with a senescent cell removal agent. The patient is administered an anti-AGE antibody conjugated to a toxin that targets and removes senescent cells.

### Example 6: Determination of Biological Age

A sample is obtained from a 50-year-old patient by buccal swab. The buccal epithelial cells and the saliva from the swab are separated. The buccal cells are trypsinized and mixed with an anti-CML monoclonal antibody. The mixture is then passed through a hemocytometer to count the senescent buccal cells. Free CML is measured by an ELISA of the saliva. The ratio of buccal cells expressing cell-surface CML to free CML in saliva is 5:1. This ratio is greater than would be expected for a healthy 50-year-old, which demonstrates that the patient has a biological age that is greater than her chronological age. The results indicate that the patient is experiencing the early onset of aging and aging-related diseases, disorders or pathological conditions due to cellular senescence. These results also indicate that the patient is in need of treatment with a senescent cell removal agent.

### Example 7: Diagnosis and Treatment of a Disease, Disorder or Pathological Condition Order Associated with Advanced Biological Aging Due to Cellular Senescence

A blood sample is obtained from a 45-year-old patient. The blood sample is centrifuged to isolate the serum. CD57+ T-cells are isolated using the Miltenyi Biotec CD8+CD57+ T Cell Isolation Kit (Bergisch Gladbach, Germany). CD57+ T-cells are counted using a hemocytometer. The serum and the isolated CD57+ T-cells are then tested for binding to an anti-CML antibody. The ratio of cells expressing cell-surface CML to free CML in serum is 10:1. This ratio indicates the patient has a biological age of 65. Since the biological age of the patient exceeds the chronological age of the patient, the patient is diagnosed with a disease, disorder or pathological condition associated with advanced biological aging due to cellular senescence. The patient is administered an anti-AGE antibody that targets and removes senescent cells.

### Example 8: In Vivo Study of the Administration of Anti-Glycation End-Product Antibody

To examine the effects of an anti-glycation end-product antibody, the antibody was administered to the aged CD1(ICR) mouse (Charles River Laboratories), twice daily by intravenous injection, once a week, for three weeks (Days 1, 8 and 15), followed by a 10 week treatment-free period. The test antibody was a commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, Minn.; catalog no. MAB3247). A control reference of physiological saline was used in the control animals.

Mice referred to as “young” were 8 weeks old, while mice referred to as “old” were 88 weeks (±2 days) old. No adverse events were noted from the administration of the antibody. The different groups of animals used in the study are shown in Table 1.

TABLE 1
The different groups of animals used in the study

| | | | | Number of Animals | |
|---|---|---|---|---|---|
| | | | | | Treatment- |
| | | | Dose Level | Main Study | Free |
| Group No. | Test Material | Mice | (μg/gm/BID/week) | Females | Females |
| 1 | Saline | young | 0 | 20 | — |
| 2 | Saline | old | 0 | 20 | 20 |
| 3 | Antibody | old | 2.5 | 20 | 20 |
| 4 | None | old | 0 | 20 | pre |
| 5 | Antibody | old | 5.0 | 20 | 20 |

— = Not Applicable, Pre = Subset of animals euthanized prior to treatment start for collection of adipose tissue.

P16^{lNK4a} mRNA, a marker for senescent cells, was quantified in adipose tissue of the groups by Real Time-qPCR. The results are shown in Table 2. In the table ΔΔCt=ΔCt mean control Group (2)−ΔCt mean experimental Group (1 or 3 or 5); Fold Expression=2^{-ΔΔCt.}

TABLE 2
P16^{INK4a} mRNA quantified in adipose tissue

| | Calculation (unadjusted to Group 4:5.59) | | | | | |
|---|---|---|---|---|---|---|
| | Group 2 vs Group 1 | | Group 2 vs Group 3 | | Group 2 vs Group 5 | |
| | Group 2 | Group 1 | Group 2 | Group 3 | Group 2 | Group 5 |
| Mean ΔCt | 5.79 | 7.14 | 5.79 | 6.09 | 5.79 | 7.39 |

| | | | |
|---|---|---|---|
| ΔΔCt | −1.35 | −0.30 | −1.60 |
| Fold Expression | 2.55 | 1.23 | 3.03 |

The table above indicates that untreated old mice (Control Group 2) express 2.55-fold more p16^{lnk4a} mRNA than the untreated young mice (Control Group 1), as expected. This was observed when comparing Group 2 untreated old mice euthanized at end of recovery Day 85 to Group 1 untreated young mice euthanized at end of treatment Day 22. When results from Group 2 untreated old mice were compared to results from Group 3 treated old mice euthanized Day 85, it was observed that p16^{lnk4a} mRNA was 1.23-fold higher in Group 2 than in Group 3. Therefore, the level of p16^{lnk4a} mRNA expression was lower when the old mice were treated with 2.5 μg/gram/BID/week of antibody.

When results from Group 2 (Control) untreated old mice were compared to results from Group 5 (5 μg/gram) treated old mice euthanized Day 22, it was observed that p16^{lnk4a} mRNA was 3.03-fold higher in Group 2 (controls) than in Group 5 (5 μg/gram). This comparison indicated that the Group 5 animals had lower levels of p16^{lnk4a} mRNA expression when they were treated with 5.0 μg/gram/BID/week, providing p16^{lnk4a} mRNA expression levels comparable to that of the young untreated mice (i.e. Group 1). Unlike Group 3 (2.5 μg/gram) mice that were euthanized at end of recovery Day 85, Group 5 mice were euthanized al: end of treatment Day 22.

These results demonstrate that the administration of the anti-AGE antibody resulted in the killing of senescent cells.

The mass of the gastrocnemius muscle was also measured, to determine the effect of antibody administration on sarcopenia. The results are provided in Table 3. The results indicate that administration of the antibody increased muscle mass as compared to controls, but only at the higher dosage of 5.0 μg/gm/BID/week.

TABLE 3
Effect of antibody administration
on mass of the gastrocnemius muscle

| | | | Weight relative to body |
|---|---|---|---|
| | Summary | Absolute weight of | mass of Gastrocnemius |
| Group | Information | Gastrocnemius Muscle | Muscle |
| 1 | Mean | 0.3291 | 1.1037 |
| | SD | 0.0412 | 0.1473 |
| | N | 20 | 20 |
| 2 | Mean | 0.3304 | 0.7671 |
| | SD | 0.0371 | 0.1246 |
| | N | 20 | 20 |
| 3 | Mean | 0.3410 | 0.7706 |
| | SD | 0.0439 | 0.0971 |
| | N | 19 | 19 |
| 5 | Mean | 0.4074 | 0.9480 |
| | SD | 0.0508 | 0.2049 |
| | N | 9 | 9 |

These results demonstrate that administration of antibodies that bind to AGEs of a cell resulted in a reduction of cells expressing p16^{lnk4a}, a biomarker of senescence. The data show that reducing senescent cells leads directly to an increase in muscle mass in aged mice. These results indicate that the loss of muscle mass, a classic sign of sarcopenia, can be treated by administration of senescent cell removal agents, such as antibodies that bind to AGEs of a cell.

This data confirms that anti-AGE antibodies are capable of selectively binding to cells expressing cell-surface AGE-modified proteins or AGE-modified peptides. Evidence of selective binding supports the suitability of anti-AGE antibodies in diagnostic applications. The data also demonstrates that anti-AGE antibodies are safe for in vivo use.

### Example 9: Affinity and Kinetics of Test Antibody

The affinity and kinetics of the test antibody used in Example 8 were analyzed using Nα,Nα-bis(carboxymethyl)-L-lysine trifluoroacetate salt (Sigma-Aldrich, St. Louis, Mo.) as a model substrate for an AGE-modified protein of a cell. Label-free interaction analysis was carried out on a BIACORE™ T200 (GE Healthcare, Pittsburgh, Pa.), using a Series S sensor chip CM5 (GE Healthcare, Pittsburgh, Pa.), with Fc1 set as blank, and Fc2 immobilized with the test antibody (molecular weight of 150,000 Da). The running buffer was a HBS-EP buffer (10 mM HEPES, 150 mM NaCl, 3 mM EDTA and 0.05% P-20, pH of 7.4), at a temperature of 25° C. Software was BIACORE™ T200 evaluation software, version 2.0. A double reference (Fc2-1 and only buffer injection), was used in the analysis, and the data was fitted to a Langmuir 1:1 binding model.

TABLE 4
Experimental set-up of affinity and kinetics analysis
Association and dissociation

| | |
|---|---|
| Flow path | Fc1 and Fc2 |
| Flow rate (μl/min.) | 30 |
| Association time (s) | 300 |
| Dissociation time (s) | 300 |
| Sample concentration (μM) | 20 - 5 - 1.25 (x2) - 0.3125 - 0.078 - 0 |

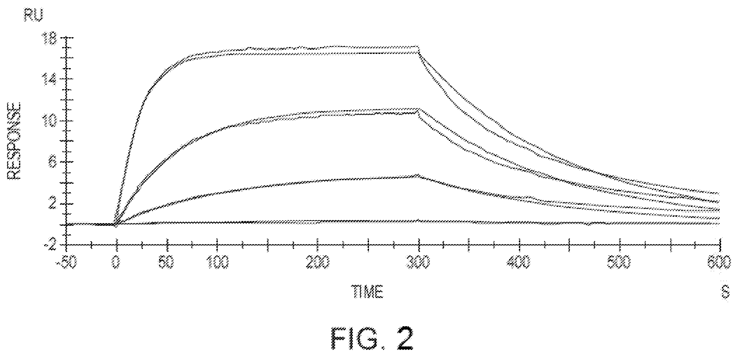

A graph of the response versus time is illustrated in FIG. 2. The following values were determined from the analysis: kₐ (1/Ms)=1.857×10³; k_{d} (1/s)=6.781×10⁻³; K_{D} (M)=3.651×10⁻⁶; Rₘₐₓ (RU)=19.52; and Chi²=0.114. Because the Chi² value of the fitting is less than 10% of Rₘₐₓ, the fit is reliable.

### Example 10: In Vivo Study of the Administration of a Carboxymethyl Lysine Monoclonal Antibody

The effect of a carboxymethyl lysine antibody on tumor growth, metastatic potential and cachexia was investigated. In vivo studies were carried out in mice using a murine breast cancer tumor model. Female BALB/c mice (BALB/cAnNCrl, Charles River) were eleven weeks old on Day 1 of the study.

4T1 murine breast tumor cells (ATCC CRL-2539) were cultured in RPMI 1640 medium containing 10% fetal bovine serum, 2 mM glutamine, 25 μg/mL gentamicin, 100 units/mL penicillin G Na and 100 μg/mL streptomycin sulfate. Tumor cells were maintained in tissue culture flasks in a humidified incubator at 37° C. in an atmosphere of 5% CO₂ and 95% air.

The cultured breast cancer cells were then implanted in the mice. 4T1 cells were harvested during log phase growth and re-suspended in phosphate buffered saline (PBS) at a concentration of 1×10⁶ cells/mL on the day of implant. Tumors were initiated by subcutaneously implanting 1×10⁵ 4 T1 cells (0.1 mL suspension) into the right flank of each test animal. Tumors were monitored as their volumes approached a target range of 80-120 mm³. Tumor volume was determined using the formula: tumor volume=(tumor width)²(tumor length)/2. Tumor weight was approximated using the assumption that 1 mm³ of tumor volume has a weight of 1 mg. Thirteen days after implantation, designated as Day 1 of the study, mice were sorted into four groups (n=15/group) with individual tumor volumes ranging from 108 to 126 mm³ and a group mean tumor volume of 112 mm³. The four treatment groups are shown in Table 5 below:

TABLE 5
Treatment groups

| | | | Dosing |
|---|---|---|---|
| Group | Description | Agent | (μg/g) |
| 1 | Control | phosphate buffered saline (PBS) | N/A |
| 2 | Low-dose | carboxymethyl lysine monoclonal | 5 |
| | | antibody | |
| 3 | High-dose | carboxymethyl lysine monoclonal | 10 |
| | | antibody | |
| 4 | Observation | None | N/A |
| | only | | |

An anti-carboxymethyl lysine monoclonal antibody was used as a therapeutic agent. 250 mg of carboxymethyl lysine monoclonal antibody was obtained from R&D Systems (Minneapolis, Minn.). Dosing solutions of the carboxymethyl lysine monoclonal antibody were prepared at 1 and 0.5 mg/mL in a vehicle (PBS) to provide the active dosages of 10 and 5 μg/g, respectively, in a dosing volume of 10 mL/kg. Dosing solutions were stored at 4° C. protected from light.

All treatments were administered intravenously (i.v.) twice daily for 21 days, except on Day 1 of the study where the mice were administered one dose. On Day 19 of the study, i.v. dosing was changed to intraperitoneal (i.p.) dosing for those animals that could not be dosed i.v. due to tail vein degradation. The dosing volume was 0.200 mL per 20 grams of body weight (10 mL/kg), and was scaled to the body weight of each individual animal.

The study continued for 23 days. Tumors were measured using calipers twice per week. Animals were weighed daily on Days 1-5, then twice per week until the completion of the study. Mice were also observed for any side effects. Acceptable toxicity was defined as a group mean body weight loss of less than 20% during the study and not more than 10% treatment-related deaths. Treatment efficacy was determined using data from the final day of the study (Day 23).

The ability of the anti-carboxymethyl lysine antibody to inhibit tumor growth was determined by comparing the median tumor volume (MTV) for Groups 1-3. Tumor volume was measured as described above. Percent tumor growth inhibition (% TGI) was defined as the difference between the MTV of the control group (Group 1) and the MTV of the drug-treated group, expressed as a percentage of the MTV of the control group. % TGI may be calculated according to the formula: % TGI=(1−MTV_{treated}/MTV_{control})×100.

The ability of the anti-carboxymethyl lysine antibody to inhibit cancer metastasis was determined by comparing lung cancer foci for Groups 1-3. Percent inhibition (% Inhibition) was defined as the difference between the mean count of metastatic foci of the control group and the mean count of metastatic foci of a drug-treated group, expressed as a percentage of the mean count of metastatic foci of the control group. % Inhibition may be calculated according to the following formula: % Inhibition=(1−Mean Count of Foci_{treated}/Mean Count of FOCi_{control})×100.

The ability of the anti-carboxymethyl lysine antibody to inhibit cachexia was determined by comparing the weights of the lungs and gastrocnemius muscles for Groups 1-3. Tissue weights were also normalized to 100 g body weight.

Treatment efficacy was also evaluated by the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study.

Statistical analysis was carried out using Prism (GraphPad) for Windows 6.07. Statistical analyses of the differences between Day 23 mean tumor volumes (MTVs) of two groups were accomplished using the Mann-Whitney U test. Comparisons of metastatic foci were assessed by ANOVA-Dunnett. Normalized tissue weights were compared by ANOVA. Two-tailed statistical analyses were conducted at significance level P=0.05. Results were classified as statistically significant or not statistically significant.

The results of the study are shown below in Table 6:

TABLE 6
Results

| | | | | | | | Gastroc. weight/ | Lung weight/ |
|---|---|---|---|---|---|---|---|---|
| | MTV | | Lung | | | | normalized | normalized |
| Group | (mm³) | % TGI | foci | % Inhibition | PR | CR | (mg) | (mg) |
| 1 | 1800 | N/A | 70.4 | N/A | 0 | 0 | 353.4/19.68 | 2799.4/292.98 |
| 2 | 1568 | 13% | 60.3 | 14% | 0 | 0 | 330.4/21.62 | 2388.9/179.75 |
| 3 | 1688 | 6% | 49.0 | 30% | 0 | 0 | 398.6/24.91 | 2191.6/214.90 |

All treatment regimens were acceptably tolerated with no treatment-related deaths. The only animal deaths were non-treatment-related deaths due to metastasis. The % TGI trended towards significance (P>0.05, Mann-Whitney) for the 5 μg/g (Group 2) and 10 μg/g treatment group (Group 3). The % Inhibition trended towards significance (P>0.05, ANOVA-Dunnett) for the 5 μg/g treatment group. The % Inhibition was statistically significant (P<0.01, ANOVA-Dunnett) for the 10 μg/g treatment group. The ability of the carboxymethyl lysine antibody to treat cachexia trended towards significance (P>0.05, ANOVA) based on a comparison of the organ weights of the lung and gastrocnemius between treatment groups and the control group. The results indicate that administration of an anti-carboxymethyl lysine monoclonal antibody is able to reduce cancer metastases.

This data confirms that anti-AGE antibodies are capable of selectively binding to cells expressing cell-surface AGE-modified proteins or AGE-modified peptides. Evidence of selective binding supports the suitability of anti-AGE antibodies in diagnostic applications. The data also demonstrates that anti-AGE antibodies are safe for in vivo use.

### Example 11: Anti-AGE Antibodies Bind to Senescent Chondrocytes In Vitro

Senescent chondrocytes were obtained from osteoarthritic joints. Anti-AGE antibodies bound to the senescent chondrocytes in vitro. These results confirm that anti-AGE antibodies are capable of binding to senescent cells. The results also confirm the suitability of anti-AGE antibodies in diagnostic applications.

### Example 12: Immunohistochemical Study

Tissue samples were obtained from patients with Alzheimer's disease and Parkinson's disease. Two Alzheimer's disease samples were taken from the hippocampus. One Parkinson's disease sample was taken from the substantia nigra, and a second Parkinson's disease sample was taken from the ventral tegmental area. All cells were stained for carboxymethyllysine (CML) using anti-AGE antibodies as described above. The Alzheimer's disease cells were stained for phosphorylated tau (phospho tau) or separately amyloid precursor protein. The Parkinson's disease cells were stained for alpha synuclein. Nuclear staining of the cells was identified using DAPI counter stain. (Experiments were carried out and images were prepared by Dr. Diego Mastroeni of Arizona State University.)

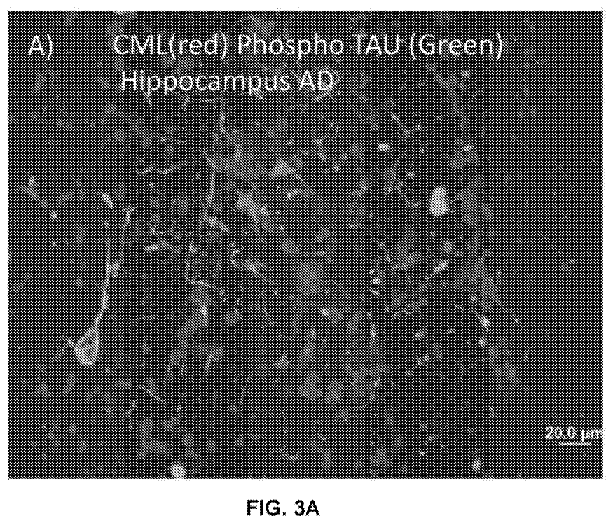

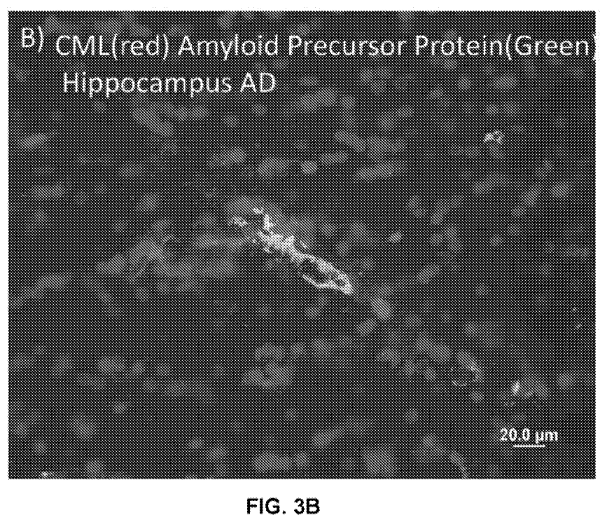

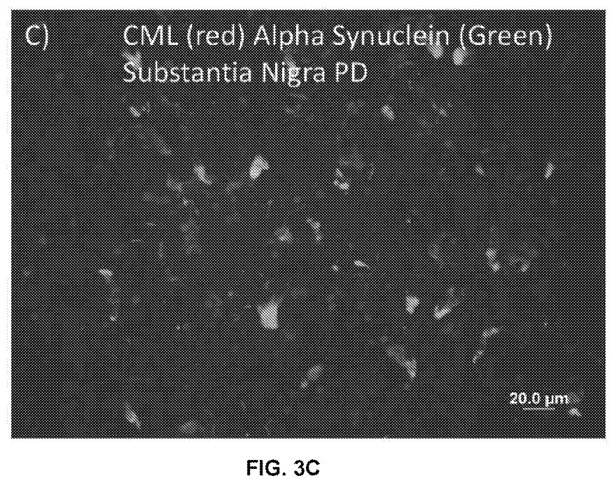

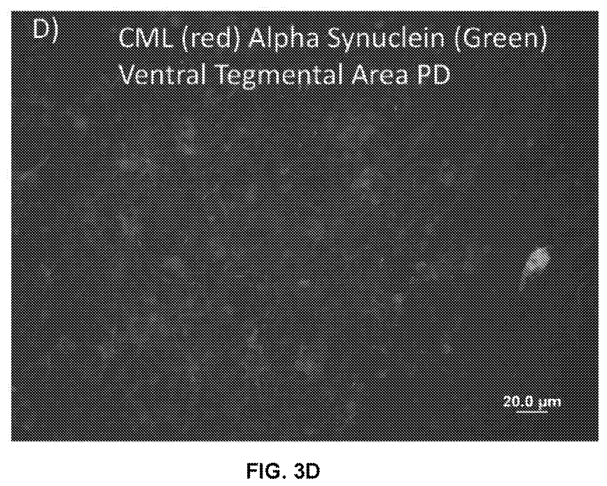

FIG. 3A is a photograph of cells of the Alzheimer's disease sample showing carboxymethyllysine stained red and phosphorylated tau stained green.

FIG. 3B is a photograph of cells of the Alzheimer's disease sample showing carboxymethyllysine stained red and amyloid precursor protein stained green.

FIG. 3C is a photograph of cells of the Parkinson's disease sample from the substantia nigra showing carboxymethyllysine stained red and alpha synuclein stained green.

FIG. 3D is a photograph of cells of the Parkinson's disease sample from the ventral tegmental area showing carboxymethyllysine stained red and alpha synuclein stained green.

CML, a well-known AGE, did not co-localize with established pathologies in Alzheimer's disease and Parkinson's disease. Instead, the CML presented on glial cells. It was suspected that the CML immunoreactivity in the Alzheimer's disease samples was with microglia, and the CML immunoreactivity in the Parkinson's disease samples was with astrocytes. The results demonstrate the presence of senescent glial cells in Alzheimer's disease and Parkinson's disease. Removal of senescent glial cells using an anti-AGE antibody would be expected to result in regeneration of the glial cells by neural stem/progenitor cells. (See, for example, Leonard, B. W. et al., “Subventricular zone neural progenitors from rapid brain autopsies of elderly subjects with and without neurodegenerative disease”, The Journal of Comparative Neurology, Vol. 515, pp. 269-294 (2009)).

This data confirms the ability of the anti-AGE antibodies to bind to AGEs present on cells in tissue samples obtained from patients with various neurodegenerative diseases. The evidence further confirms the suitability of anti-AGE antibodies in diagnostic applications.

### REFERENCES

1. Lowe, R. et al., “Buccals are likely to be a more informative surrogate tissue than blood for epigenome-wide association studies”, Epigenetics, Vol. 8, No. 4, pp. 445-454 (2013).
2. Kared, H. et al., “CD57 in human natural killer cells and T-lymphocytes”, Cancer Immunology, Immunotherapy, Vol. 65, No. 4, pp. 441-452 (2016).
3. Yoon, M-S. et al., “Characterisation of advanced glycation endproducts in saliva from patients with diabetes mellitus”, Biochemical and Biophysical Research Communications, Vol. 323, Issue 2, pp. 377-381 (2004).
4. Pearce, M. S. et al., “Childhood growth, IQ and education as predictors of white blood cell telomere length at age 49-51 years: the Newcastle Thousand Families Study”, PLoS ONE, Vol. 7, Issue 7, e40116 (2012).
5. “Telomere Testing White Paper”, Titanovo, available online at titanovo.com/telomere-length-testing/ (accessed on Apr. 20, 2017).
6. “Carboxymethyl Lysine (CML) ELISA Cat. No. KT-32428”, Kamiya Biomedical Company, available online at www.kamiyabiomedical.com/pdf/KT-32428.pdf (accessed on Apr. 20, 2017).
7. “CD8+CD57+ T Cell Isolation Kit Data Sheet”, Miltenyi Biotec, available online at www.miltenyi biotec. com/˜/media/Images/Products/Import/0001600/IM0001652.ashx?force=1 (2008).
8. “CD8+CD57+ T Cell Isolation Kit, human”, Miltenyi Biotec, available online at www.miltenyibiotec.com/en/products-and-services/macs-cell-separation/cell-separation-reagents/t-cells/cd8-cd57-t-cell-isolation-kit-human aspx (accessed on Apr. 19, 2017).
9. “Extraction of genomic DNA from buccal epithelial cells”, available online at authors.fhcrc.org/591/1/Buccal %20Cell%20DNA %20lsol%20v2.pdf (accessed on Apr. 19, 2017).
10. Severin, F. F. et al., “Advanced glycation of cellular proteins as a possible basic component of the ‘Master Biological Clock’”, Biochemistry (Moscow), Vol. 78, No. 9, pp. 1331-1336 (2013).
11. Wong, R. et al., “Use of RT-PCR and DNA microarrays to characterize RNA recovered by non-invasive tape harvesting of normal and inflamed skin”, The Journal of Investigative Dermatology, Vol. 123, pp. 159-167 (2004).
12. Yang, S. et al., “Impact of oxidative stress biomarkers and carboxymethyllysine (an advanced glycation end product) on prostate cancer: a prospective study”, Clinical Genitourinary Cancer, Vol. 13, No. 5, pp. e347-e351 (2015).
13. Meerwaldt, R. et al., “Skin autofluorescence is a strong predictor of cardiac mortality in diabetes”, Diabetes Care, Vol. 30, No. 1, pp. 107-112 (2007).
14. Cosgrove, C. et al., “The impact of 6-month training preparation for an Ironman triathlon on the proportions of naïve, memory and senescent T cells in resting blood”, European Journal of Applied Physiology, Vol. 112, No. 8, pp. 2989-2998 (2012).
15. Krtolica, A. et al., “Senescent fibroblasts promote epithelial cell growth and tumorigenesis: a link between cancer and aging”, Proceedings of the National Academy of Sciences, Vol. 98, No. 21, pp. 12072-12077 (2001).
16. Schmid, D. et al., “Collage glycation and skin aging”, Cosmetics and Toiletries Manufacture Worldwide, available online at mibellebiochemistry.com/app/uploads/2015/03/GSP-T-for-Skin_Collagen-glycation-and-skin-aging-CT-2002.pdf (accessed on Apr. 27, 2017).
17. Spielmann, G. et al., “Aerobic fitness is associated with lower proportions of senescent blood T-cells in man”, Brain, Behavior and Immunity, Vol. 25, No. 8, pp. 1521-1529 (2011).
18. Pauwels, E. K. J. et al., “Radiolabelled monoclonal antibodies: a new diagnostic tool in nuclear medicine”, Radiotherapy and Oncology, Vol. 1, No. 4, pp. 333-338 (1984).
19. U.S. Pat. App. Pub. No. US 2016/0017033.
20. Khoja, L. et al., “A pilot study to explore circulating tumour cells in pancreatic cancer as a novel biomarker”, British Journal of Cancer, Vol. 106, No. 3, pp. 508-516 (2012).
21. Rüster, M. et al., “Detection of elevated NE-carboxymethyllysine levels in muscular tissue and in serum of patients with fibromyalgia”, Scandinavian Journal of Rheumatology, Vol. 34, No. 6, pp. 460-463 (2005).
22. Al-Abed, Y. et al., “NE-Carboxymethyllysine formation by direct addition of glyoxal to lysine during the Maillard reaction”, Bioorganic & Medicinal Chemistry Letters, Vol. 5, No. 18, pp. 2161-2162 (1995).
23. Freise, A. C. et al., “In vivo imaging with antibodies and engineered fragments”, Molecular Immunology, Vol. 67, No. 2, pp. 142-152 (2015).
24. Wendel, U. et al., “A novel monoclonal antibody targeting carboxymethyllysine, an advanced glycation end product in atherosclerosis and pancreatic cancer”, PLoS One, Vol. 13, No. 2, e0191872 (2018).

What is claimed is:

1. A method of diagnosing and treating a disease, disorder or pathological condition associated with cellular senescence in a patient, comprising:
obtaining a sample from the patient;
measuring the number of cells that exhibit cell-surface AGEs in the sample; and
diagnosing the patient with a disease, disorder or pathological condition associated with cellular senescence when the number of cells that exhibit cell-surface AGEs in the sample is greater than the number of cells that exhibit cell-surface AGEs in a control; and
administering an effective amount of a senescent cell removal agent to the diagnosed patient.

2. A method of determining the biological age of a patient, comprising:
obtaining a sample from a patient containing cells and non-cellular material;
separating the cells from the non-cellular material;
measuring the number of cells that exhibit cell-surface AGEs in the sample by contacting the cells with an anti-AGE antibody and detecting binding between cell-surface AGEs and the anti-AGE antibody;
measuring the number of unbound AGEs in the sample by contacting the non-cellular material with an anti-AGE antibody and detecting binding between unbound AGEs and the anti-AGE antibody;
comparing the ratio of cell-surface AGEs to unbound AGEs in the sample; and
administering a therapeutically effective amount of a senescent cell removal agent to the patient if the biological age of the patient exceeds the chronological age of the patient.

3. A method of diagnosing and treating a disease, disorder or pathological condition associated with advanced biological aging due to cellular senescence in a patient, comprising:
obtaining a sample from a patient containing cells and non-cellular material;
separating the cells from the non-cellular material;
measuring the number of cells that exhibit cell-surface AGEs in the sample by contacting the cells with an anti-AGE antibody and detecting binding between cell-surface AGEs and the anti-AGE antibody;
measuring the number of unbound AGEs in the sample by contacting the non-cellular material with an anti-AGE antibody and detecting binding between unbound AGEs and the anti-AGE antibody;
comparing the ratio of cell-surface AGEs to unbound AGEs in the sample to determine the biological age of the patient;
diagnosing the patient with a disease, disorder or pathological condition associated with advanced biological aging due to cellular senescence when the biological age of the patient exceeds the chronological age of the patient; and
administering an effective amount of a senescent cell removal agent to the diagnosed patient.

4. The method of claim 1, further comprising:
determining the biological age of the patient by comparing the number of cells that exhibit cell-surface AGEs in the sample to the number of cells that exhibit cell-surface AGEs in an age-matched control, after the measuring.

5. A method of detecting AGE-modified cells in a subject in vivo, comprising:
administering to the subject an anti-AGE antibody that has been labeled with a detectable label.

6. A kit for detecting cells expressing cell surface advanced glycation end-products, comprising,
an anti-AGE antibody,
a control sample, and
optionally, a reagent that binds to the anti-AGE antibody.

7. The method of claim 1, wherein the sample is selected from the group consisting of a saliva sample, a buccal swab, a blood sample, a skin sample, and a urine sample.

8. The method of claim 2, wherein the cells are selected from the group consisting of T-cells, erythrocytes, fibroblasts, and epithelial cells.

9. The method of claim 3, wherein the measuring comprises an ELISA, cell sorting, or cell counting.

10. The method of claim 1, wherein the diagnosing comprises diagnosing the patient with a disease, disorder or pathological condition associated with cellular senescence when at least 5% of the cells in the sample exhibit cell-surface AGEs.

11. (canceled)

12. The method of claim 1, wherein the disease, disorder or pathological condition associated with cellular senescence comprises at least one disease, disorder or pathological condition selected from the group consisting of Alzheimer's disease, amyotrophic lateral sclerosis, chronic obstructive pulmonary disease, Huntington's chorea, idiopathic pulmonary fibrosis, muscular dystrophy, macular degeneration, cataracts, diabetic retinopathy, Parkinson's disease, progeria, vitiligo, cystic fibrosis, atopic dermatitis, eczema, arthritis, atherosclerosis, cancer and metastatic cancer, cancer therapy-related disability or cancer therapy side effects, hypertension, glaucoma, osteoporosis, sarcopenia, cachexia, stroke, myocardial infarction, atrial fibrillation, transplantation rejection, diabetes mellitus—Type I, diabetes mellitus—Type II, radiation exposure, HIV treatment side effects, chemical weapons exposure, poisoning, inflammation, nephropathy, Lewy body dementia, prion disease, lordokyphosis, auto-immune disorders, loss of adipose tissue, psoriasis, Crohn's disease, asthma, the physiological effects of aging, idiopathic myopathy, multiple sclerosis, neuromyelitis optica, epilepsy and adrenoleukodystrophy.

13. The method of claim 2, wherein the anti-AGE antibody binds to a cell-surface protein or peptide that exhibits an AGE modification selected from the group consisting of FFI, pyrraline, AFGP, ALI, carboxymethyllysine, carboxyethyllysine and pentosidine.

14-16. (canceled)

17. The method of claim 3, wherein the patient is selected from the group consisting of humans, rats, goats, sheep, cows, horses, camels, dogs, and cats.

18. (canceled)

19. The method of claim 5, further comprising exposing the subject to an apparatus that is capable of detecting the label.

20. The method of claim 5, wherein the AGE-modified cells are senescent cells.

21. The method of claim 5, wherein the AGE-modified cells are metastatic cancer cells.

22. The method of claim 5, wherein the label comprises at least one label selected from the group consisting of radiolabels, fluorescent labels, gold nanoparticles, gadolinium nanoparticles, positron emitters, dyes that emit in near infrared, quantum dots, superparamagnetic iron oxide, carbon nanotubes, and microbubbles.

23. (canceled)

24. The kit of claim 6, further comprising the reagent that binds to the anti-AGE antibody,
wherein the reagent comprises a secondary antibody.

25-26. (canceled)

27. The kit of claim 24, wherein the anti-AGE antibody and/or the reagent are labelled, and
the label comprises at least one label selected from the group consisting of a fluorescent label, a radiolabel, and a gold particle.

28-29. (canceled)

30. The method of claim 1, wherein the senescent cell removal agent comprises a therapeutic anti-AGE antibody, an anti-AGE antibody conjugated to a toxin, a senolytic agent, and combinations thereof.

31-33. (canceled)
